(19) United States
(12) Patent Application Publication
(10) Pub. No.: US 2016/0354620 A1
(43) Pub. Date: Dec. 8, 2016

(54) RESPIRATORY MASKS FOR USE IN AIRCRAFTS

(71) Applicant: AIRBUS GROUP INDIA PRIVATE LIMITED, Bangalore (IN)

(72) Inventor: ANURAG SHARMA, Bangalore (IN)

(21) Appl. No.: 15/166,351

(22) Filed: May 27, 2016

(30) Foreign Application Priority Data
Jun. 2, 2015 (IN) 2778/CHE/2015

(51) Int. Cl. A62B 7/14; A61B 5/08; B64D 10/00; A62B 18/10; A62B 7/10; A62B 18/08; A62B 18/02; A61B 5/145

(52) U.S. Cl. CPC A62B 7/14; A62B 18/02; A61B 5/0816; A61B 5/082; A61B 5/14542; A62B 18/10; A62B 7/10; A62B 18/084; B64D 10/00

(57) ABSTRACT

A respiratory mask for use in an aircraft is disclosed. In one embodiment, the respiratory mask includes a plurality of sensors for monitoring at least one of cockpit air for parameters capable of affecting oxygen level and health of a crew member health for parameters capable of causing respiratory disorder, and providing associated output signals. Further, the respiratory mask includes a regulator electronically coupled to the sensors. The regulator automatically switches between operating modes to supply respiratory gas to the crew member based on the associated output signals of the plurality of sensors. The operating modes may include a dilution mode, an emergency mode, and a recirculation mode.

### RELATED APPLICATIONS

Benefit is claimed under 35 U.S.C. 119(a)-(d) to Foreign Application Serial No. 2778/CHE/2015 filed in India entitled “RESPIRATORY MASKS FOR USE IN AIRCRAFTS”, filed on Jun. 2, 2015 by AIRBUS GROUP INDIA PRIVATE LIMITED which is herein incorporated in its entirety by reference for all purposes.

A reference is made to an U.S. application Ser. No. 12/748,473 filed on Mar. 29, 2010 and entitled “Adaptable demand dilution oxygen regulator for use in aircrafts” and an U.S. application Ser. No. 13/645,519 filed on Oct. 5, 2012 and entitled “Adaptable demand dilution oxygen regulator for use in aircrafts”. A reference is also made to an U.S. application Ser. No. 13/390,517 filed on Feb. 14, 2012 and entitled “Adaptable oxygen regulator system and method with an electronic control device”.

### TECHNICAL FIELD

Embodiments of the present subject matter generally relate to respiratory masks, and more particularly, to respiratory masks for use in aircrafts.

### BACKGROUND

Crew members in a cockpit of an aircraft may suffer from several dangerous and catastrophic emergencies. For example, the crew members may suffer from respiratory related emergencies, such as hyperventilation and hypoxia. These emergencies may occur due to smoke in the cockpit, reduction in pressure level due to height of the aircraft, and the like. For example, smoke in the cockpit may occur due to short circuit, equipment failure, insulation breakdown, and the like.

Typically, during such emergencies, the crew member may be provided with a respiratory mask and further, the crew member may have to manually operate a regulator of the respiratory mask to obtain the needed respiratory gases for a smooth respiration. Manual operation of the regulator may not be possible in emergencies, such as smoky environments, due to heavy workload and/or difficulty in locating switches for suppressing the smoke. Also, manual operation of the regulator in such emergencies may need crew member's attention, which may result in distracting the crew member from other needed vital operations.

### SUMMARY

A respiratory mask for use in an aircraft is disclosed. According to one aspect of the present subject matter, the respiratory mask may include a plurality of sensors for monitoring either cockpit air for parameters capable of affecting oxygen level and/or health of a crew member for parameters capable of causing respiratory disorder. Further, the plurality of sensors may provide associated output signals. Furthermore, the respiratory mask may include a regulator electronically coupled to the sensors for automatically switching between operating modes to supply the respiratory gas to the crew member based on the associated output signals of the sensors. The operating modes may include a dilution mode, an emergency mode and a recirculation mode.

According to another aspect of the present subject matter, information associated with either cockpit air for parameters capable of affecting oxygen level and/or health of a crew member for parameters capable of causing respiratory disorder is received. Further, switching between operating modes to supply a respiratory gas to the crew member is automatically performed based on the information received. The operating modes may include a dilution mode, an emergency mode and a recirculation mode.

The respiratory mask and method disclosed herein may be implemented in any means for achieving various aspects. Other features will be apparent from the accompanying drawings and from the detailed description that follow.

### BRIEF DESCRIPTION OF THE DRAWINGS

Various embodiments are described herein with reference to the drawings, wherein:

FIG. 1 is a block diagram showing major components of an example respiratory mask for use in an aircraft and their connectivity;

FIG. 2 is a perceptive view of an example respiratory mask for use in an aircraft, such as those shown in FIG. 1;

FIG. 3 is a flow diagram illustrating a method for automatically switching between operating modes of a respiratory mask, in accordance with the present subject matter.

The drawings described herein are for illustration purposes only and are not intended to limit the scope of the present disclosure in any way.

### DETAILED DESCRIPTION

In the following detailed description of the embodiments of the present subject matter, references are made to the accompanying drawings that form a part hereof, and in which are shown by way of illustration specific embodiments in which the present subject matter may be practiced. These embodiments are described in sufficient detail to enable those skilled in the art to practice the present subject matter, and it is to be understood that other embodiments may be utilized and that changes may be made without departing from the scope of the present subject matter. The following detailed description is, therefore, not to be taken in a limiting sense, and the scope of the present subject matter is defined by the appended claims.

Generally, a crew member of an aircraft is provided with a respiratory mask for obtaining needed respiratory gas during emergencies. Such emergencies may include smoke in a cockpit of the aircraft, pressurization loss, air contamination, and the like. However, such respiratory masks may be generally bulky and removal of the respiratory mask from a stowage located beside a seat of the crew member for wearing it may be cumbersome and may need significant time. During this process, the crew member may ends up inhaling the smoke.

Embodiments described herein provide a respiratory mask for use by the crew member in an aircraft. The crew member may include a pilot, flight attendant, flight medic, and the like. In an embodiment, the respiratory mask may include a plurality of sensors for monitoring either cockpit air for parameters capable of affecting oxygen level and/or crew member's health for parameters capable of causing respiratory disorder. The parameters capable of affecting oxygen level may be understood as presence of smoke in a cockpit of the aircraft, pressure level inside the cockpit, and contaminants present in cockpit ambient air. Similarly, the parameters capable of causing respiratory disorder may be breathing rate of a crew member in the aircraft, carbon dioxide level present in exhaled gas of the crew member, partial pressure of oxygen present in crew member's blood, and tissue oxygen saturation of the crew member. Further, the sensors may provide associated output signals. Furthermore, the respiratory mask may include a regulator electronically coupled to the sensors for automatically switching between operating modes to supply the respiratory gas to the crew member based on the associated output signals of the sensors. For example, the operating modes may include a dilution mode, an emergency mode and a recirculation mode. The respiratory gas may include oxygen during the emergency mode, a combination of the exhaled gas including carbon dioxide (CO₂) and gases already present in the mouthpiece and regulator cavity during the recirculation mode, and a combination of air inside the cockpit or air mixed with oxygen during the dilution mode.

Thus, the respiratory mask functions continuously as a respiratory aid during long haul crew member operations to prevent sudden incapacitation due to the emergencies and preserves bottled oxygen supply in the aircraft. Also, the respiratory mask is lightweight. Therefore, the mask can be worn by the crew member comfortably throughout the flight.

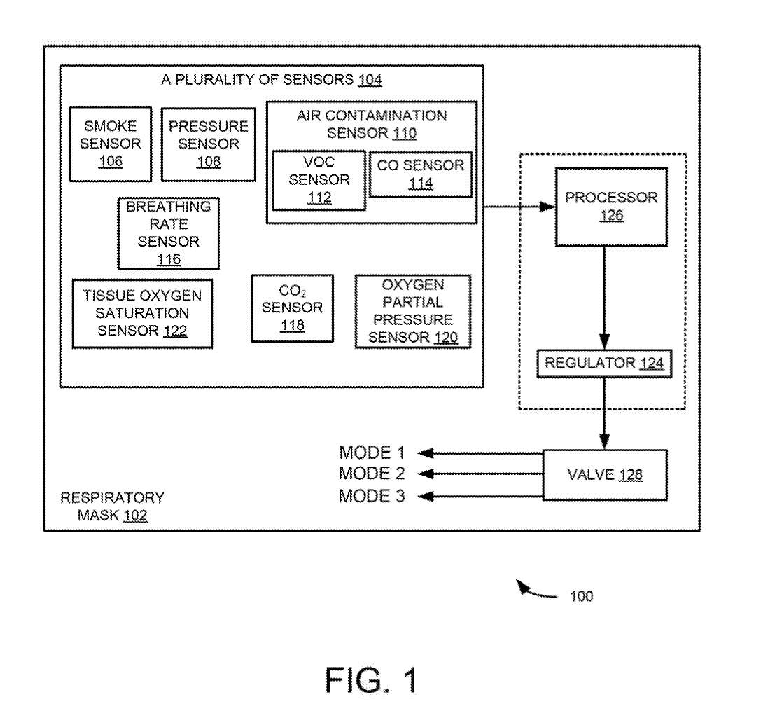

The respiratory mask 102, in accordance with an example of the present subject matter, is explained in detail with reference to FIGS. 1 to 3, where the reference numerals indicate key components of the respiratory mask 102.

FIG. 9 is a block diagram 100 of a respiratory mask 102 for use in an aircraft (not shown in the figures), according to one embodiment. The respiratory mask 102 may be worn by a crew member comfortably throughout the flight of the aircraft. For example, the crew member may include a pilot, flight attendant, flight medic, and the like. In addition, the respiratory mask 102 may be operable throughout the flight of the aircraft for protecting the crew member from several dangerous and catastrophic emergencies, thereby avoiding hazardous situations such as crash of the aircraft. For example, the emergencies may be respiratory related emergencies, such as hyperventilation, hypoxia and the like.

In accordance with the present subject matter, the respiratory mask 102 may further include a regulator 124 and a plurality of sensors 104. In one implementation, the regulator 124 may be electronically coupled to the plurality of sensors 104 through a processor 126. In one implementation, the regulator 124 and the processor 126 may be separate components coupled to each other. In another implementation, the regulator 124 may include the processor 126. Further, the plurality of sensors 104 may include smoke sensor 106 pressure sensor 108, air contamination sensor 110, breathing rate sensor 116, tissue oxygen saturation sensor 122, carbon dioxide (CO₂) sensor 118, and oxygen partial pressure sensor 120. In one example, the air contamination sensor 110 may include Volatile Organic Compound (VOC) sensor 112 and Carbon monoxide (CO) sensor 114. The processor 126 receives output of the plurality of sensors 104 as an input. The output of the plurality of sensors 104 is then processed by the processor 126 and sent to the regulator 124. The regulator 124, upon receiving the processed output, actuates one or more valves 128 for switching either to mode 1 or mode 2 or mode 3 based on the received processed output. In one embodiment, the one or more valves 128 may reside inside the regulator 124. For the purpose of simplicity of explanation, the mode 1 may be understood as an emergency mode, the mode 2 may be understood as a recirculation mode, and the mode 3 may be understood as a dilution mode. Further, actuation of the one or more valves 128 to switch either to mode 1 or mode 2 or mode 3 may help to supply a respiratory gas to the crew member. The respiratory gas may include oxygen which may be supplied during the emergency mode. The respiratory gas may also include the exhaled gas which may be supplied during the recirculation mode. The respiratory gas may further include a combination of air inside the cockpit or the cockpit ambient air progressively mixed with more oxygen as altitude increases may be supplied during the dilution mode.

Further, the regulator 124 may actuate the one or more valves 128 based on the output of the sensors 104. In one example, the regulator 124 may actuate the one or more valves 128 when the output indicates emergency conditions. The emergency conditions may be understood as conditions when there is presence of smoke in the cockpit, the pressure level present inside the cockpit is lower than a predetermined pressure level, the partial pressure of the oxygen present in the crew member's blood is lower than a predetermined partial pressure of the oxygen, the carbon dioxide level present in the exhaled gas is higher than a predetermined carbon dioxide level, and/or the breathing rate of the crew ember is deviated from a predetermined breathing rate.

The predetermined pressure level may be understood as a level of the pressure inside the cockpit at which the crew member can breathe comfortably without any hurdle. For example, the predetermined pressure level may be considered as a level of the pressure at a height of 6000 feet from the sea level. Similarly, the predetermined partial pressure of the oxygen may be understood as a level of the partial pressure of the oxygen at which mental acuity of the crew member may start to be affected. For example, the predetermined partial pressure of the oxygen may be in between 94% to 95%. In a like manner, for the purpose of simplicity of the explanation, the predetermined carbon dioxide level may be understood as a level of the carbon dioxide present in the exhaled gas from the body of the crew member such that the respiratory system of the crew member is healthy. Likewise the predetermined breathing rate may be understood as breathing rate at which the crew member breathes comfortably. The breathing rate may be understood as, for example, number of breaths per minute. Alternatively, the breathing rate may be understood, for example, as an expiration volume in liter/sec or an expiration volume per breath. The mode of the respiratory mask 102 in accordance with this implementation may be understood as emergency mode. Such above explained emergency conditions may result in lack of oxygen and the crew member may get respiratory disorder, such as hypoxia, due to lack of oxygen which may lead to unconsciousness of the crew member.

To overcome the respiratory disorder due to the lack of oxygen, the crew member may be forced to inhale oxygen so that the level of oxygen in the blood can be retained to a level where the crew member can breathe smoothly. Therefore, the regulator 124 enables providing oxygen by actuating the one or more valves 128. Thus, inhalation of oxygen may protect the crew member from hypoxia caused by the above explained emergency conditions. Protection of the crewmember using oxygen is not limited only to hypoxia and inhalation of oxygen may also protect the crew member from the respiratory disorders other than hypoxia.

In another example the regulator 124 may actuate the one or more valves 128 when the output indicates that a percentage of oxygen present in the blood of the crew member is at least 94%. The regulator 124 may also actuate the one or more valves 128 when the carbon dioxide level present in the exhaled gas is lower than the predetermined carbon dioxide level and/or when the breathing rate of the crew member is higher than the predetermined breathing rate and/or expiration volume is low which may occur during a high stress condition. In such above explained conditions, the crew member may be hyperventilating and may subsequently lead to unconsciousness of the crew member. The mode of the respiratory mask 102 in accordance with this implementation may be understood as recirculation mode. Therefore it is essential to protect the crew members from these conditions. To overcome the respiratory disorder caused by these conditions, the crew member may be momentarily forced to inhale the exhaled gas till the breathing rate can be controlled. The oxygen may be stored in an oxygen storage kept onboard for supplying when needed. Further, inhalation of the recirculated gas helps the brain to auto-regulate the breathing rate, thereby protecting the crew member from hyperventilation caused by the above explained conditions.

In yet another example, the regulator 124 may actuate the one or more valves 128 to the dilution mode when the output of the sensors 104 indicates any one of the conditions such as presence of no smoke in the cockpit, the pressure level inside the cockpit is higher than a predetermined pressure level, the partial pressure of the oxygen present in the crew member's blood is higher than a predetermined partial pressure of the oxygen and/or the breathing rate of the crew member is equal to a predetermined breathing rate. The predetermined breathing rate, the predetermined carbon dioxide level, and the predetermined partial pressure of the oxygen may be understood as explained above. The dilution mode may be understood as a mode where the respiratory system of the crew member functions normally in which there is no symptoms of either hyperventilation or hypoxia or any other respiratory disorder in the crew member.

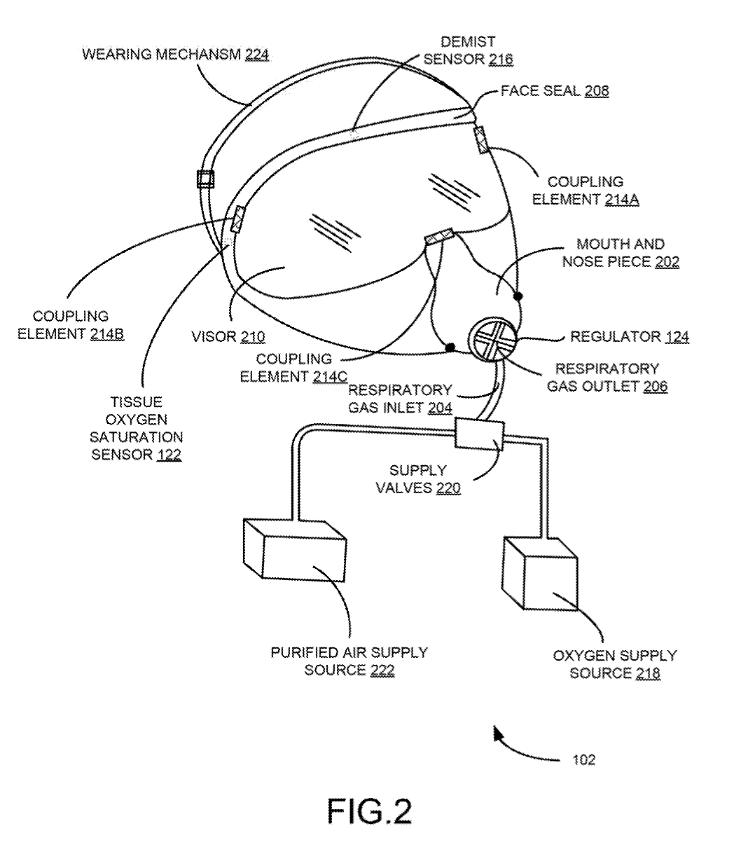

Referring now to FIG. 2 which illustrates a perspective view of a respiratory mask 102 for use in an aircraft, in accordance with an example of the present subject matter. As illustrated in FIG. 2, the respiratory mask 102 may include a mouth and nose piece 202 connected to a regulator 124 similar to as illustrated in FIG. 1. The mouth and nose piece 202 may have smoke sensors (similar to as illustrated in FIG. 1) for sensing presence of smoke in a cockpit of the aircraft and may also have pressure sensors (similar to as illustrated in FIG. 1) for sensing the pressure level present inside the cockpit. The smoke sensors and the pressure sensors may be mounted, for example, around the mouth and nose piece 202. For example, the smoke sensors may be mounted on the regulator 124. The smoke sensors are explained to be mounted on the regulator 124 for the purpose of explanation and can be mounted anywhere on the respiratory mask 102 or can be mounted anywhere in the cockpit for sensing the smoke in the cockpit. Further, the respiratory mask 102 may include a respiratory gas inlet 204 and a respiratory gas outlet 206. The respiratory gas inlet 204 and a respiratory gas outlet 206 may be connected to the mouth and nose piece 202 for the purpose of inhaling the respiratory gas and exhaling the exhaled gas respectively. The respiratory gas inlet 204 may include breathing rate sensors (similar to as illustrated in FIG. 1) disposed therein for sensing a breathing rate of a crew member in the aircraft. Similarly, the respiratory gas outlet 206 may include carbon dioxide sensors (similar to as illustrated in FIG. 1) disposed therein for sensing a carbon dioxide level in an exhaled gas by the crew member. Further, the respiratory mask 102 may include a microphone (not shown in the figures) for the communication purpose. For example, the microphone may be attached to the mouth and nose piece 202. In another example, the microphone may be inbuilt with the mouth and nose piece 202.

Further, the respiratory mask 102 as illustrated in FIG. 2 may include a peripheral face seal 208. The peripheral face seal 208 may be fitted to a visor 210. The visor 210 is removably attached to the respiratory mask 102 with the help of coupling elements 214A 214B, and 214C. Further, the peripheral face seal 208 enables to airtight the face of the crew member for protecting the face from contaminates present in air of cockpit and heat generated by fumes or smoke. The peripheral face seal 208 may be made of, for example, pliable/compliant materials. Furthermore, the peripheral face seal 210 may include a demist sensor 216 disposed thereon, for sensing the mist on the visor 210. The demist sensor 216 may be communicatively coupled to the regulator 124 for providing a feedback to the regulator 124 that there is a mist on the visor 210 and hence demisting is needed. The regulator 124, upon receiving the feedback, may perform demisting to clear the visor 210 by opening its outflow outlet to reduce outlet pressure and increasing ventilation flow.

Further, the respiratory mask 102 may be connected to an oxygen supply source 218 through one or more supply valves 220. The oxygen supply source 218 may be utilized to deliver oxygen to the crew member when there is demand for oxygen, such as during emergency mode and recirculation mode. For delivering oxygen to the crew ember, the regulator 124 may actuate the one or more valves 128 to enable flow of oxygen from the oxygen supply source 218 to the crew member through the respiratory gas inlet 204 and the mouth and nose piece 202. During emergency mode, the respiratory gas may include 100% oxygen supplied from the oxygen supply source 218. Whereas, during recirculation mode, the respiratory gas may include a combination of oxygen from the oxygen supply source 218 and exhaled gas by the crew member.

Furthermore, in accordance with an embodiment of the present subject matter, the respiratory mask 102 may be connected to a purified air supply source 222 through the one or more supply valves 220. The purified air supply source 222 may be utilized to supply purified air to the crew member when air inside the cockpit is contaminated by contaminants, such as Volatile Organic Compounds (VOC) and Carbon Monoxide (CO). The purified air supply source 222 may purify the air inside the cockpit to eliminate contaminants from the air and generate a purified air. The purified air is then supplied to the crew member by the purified air supply source 222 through the respiratory gas inlet 204 and the mouth and nose piece 202. In one implementation, the purified air is supplied during the dilution mode which preserves consumption of oxygen from the oxygen storage. In accordance with another embodiment of the present subject matter, the respiratory mask 102 may include a filter unit (not shown the figures) in the mouth and nose piece 202 to filter the air inside the cockpit and provide purified/filtered air to the crew member. In one embodiment, the respiratory mask 102 may also include a wearing mechanism 224 for facilitating the crew member to wear the respiratory mask 102. The wearing mechanism 224, for example, may be straps connected to each other in such a manner that they can be utilized for wearing the respiratory mask 102. In one example, the wearing mechanism 224 may be an adjustable strap or band which can be fitted around the head of the crew member to wear the respiratory mask 102.

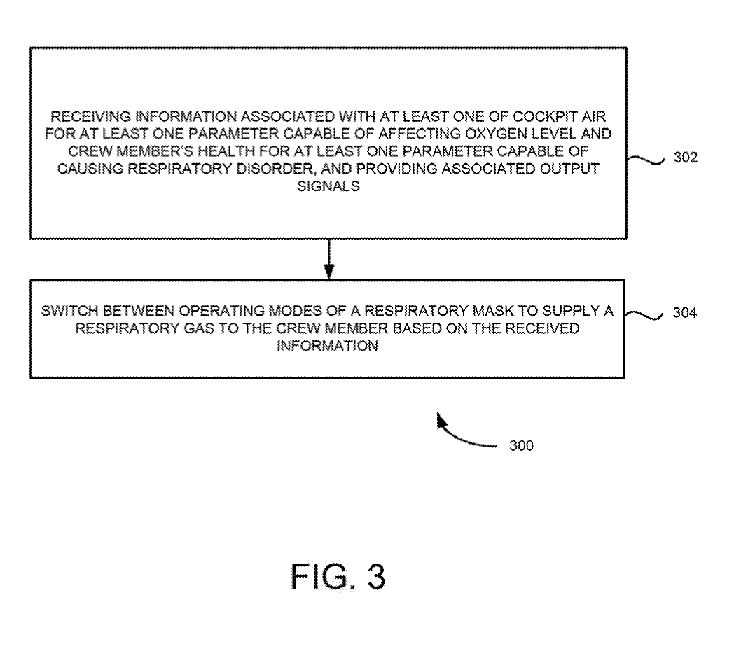

Referring now to FIG. 3 which is a flow diagram 300 illustrating an example method, in accordance with the present subject matter. At step 302, information associated with either cockpit air for parameters capable of affecting oxygen level and/or crew member's health for parameters capable of causing respiratory disorder is received. The parameters capable of affecting oxygen level may be presence of smoke in a cockpit of the aircraft, pressure level inside the cockpit, and contaminants present in cockpit ambient air. Similarly, the parameters capable of causing respiratory disorder may be breathing rate of a crew member in the aircraft, carbon dioxide level present in exhaled gas of the crew member, partial pressure of oxygen present in crew member's blood, and tissue oxygen saturation of the crew member.

At step 304, automatic switching between various operating modes is performed based on the received information. The operating modes may include a dilution mode, an emergency mode, and a recirculation mode. The dilution mode, the emergency mode, and the recirculation ode may be understood as explained above.

The respiratory mask 102 in accordance with the present subject matter may include the visor 210 either covering full face of the crew member or only the eyes of the crew member. The respiratory mask 102 may start functioning when it is removed from a stowage located beside a seat of the crew member. Further, respiratory mask 102 may be wearable during entire flight time due to its light weight. Furthermore, the respiratory mask 102 may be capable to operate in either emergency mode or recirculation mode or dilution mode which reduces consumption of oxygen from the oxygen storage and hence enables the crew member to wear the respiratory mask 102 during entire flight time without any interruption in the supply of the respiratory gas.

It may be noted that the above-described examples of the present solution is for the purpose of illustration only. Although the solution has been described in conjunction with a specific, embodiment thereof, numerous modifications may be possible without materially departing from the teachings and advantages of the subject matter described herein. Other substitutions, modifications and changes may be made without departing from the spirit of the present solution. All of the features disclosed in this specification (including any accompanying claims, abstract and drawings), and/or all of the steps of any method or process so disclosed, may be combined in any combination, except combinations where at least some of such features and/or steps are mutually exclusive.

The terms “include,” “have,” and variations thereof, as used herein, have the same meaning as the term “comprise” or appropriate variation thereof. Furthermore, the term “based on” as used herein, means “based at least in part on.”

The present description has been shown and described with reference to the foregoing examples. It is understood, however, that other forms, details, and examples can be made without departing from the spirit and scope of the present subject matter that is defined in the following claims.

What is claimed is:

1. A respiratory ask for use in an aircraft, comprising:
a plurality of sensors monitoring at least one of cockpit ambient air for at least one parameter capable of affecting oxygen level and health of a crew member for at least one parameter capable of causing respiratory disorder, and providing associated output signals; and
a regulator electronically coupled to he plurality of sensors, wherein the regulator automatically switches between operating modes to supply respiratory gas to the crew member based on the associated output signals of the plurality of sensors, wherein the operating modes comprise a dilution mode, an emergency mode, and a recirculation mode.

2. The respiratory mask of claim 1, wherein the at least one parameter capable of affecting oxygen level is selected from the group consisting of presence of smoke in a cockpit of the aircraft, pressure level inside the cockpit, and contaminants present in the cockpit ambient air.

3. The respiratory mask of claim 1, wherein the at least one parameter capable of causing respiratory disorder is selected from the group consisting of breathing rate of the crew member in the aircraft carbon dioxide level present in exhaled gas of the crew member, partial pressure of oxygen present in blood of the crew member, and tissue oxygen saturation of the crew member.

4. The respiratory mask of claim 1, wherein the regulator is configured to:
automatically switch to the emergency mode when at least one emergency condition occurs, wherein the at least one emergency condition is selected from the group consisting of presence of smoke in a cockpit, pressure level inside the cockpit is lower than a predetermined pressure level, partial pressure of oxygen present in blood of the crew member is lower than a predetermined partial pressure of oxygen, carbon dioxide level present in exhaled gas is higher than a predetermined carbon dioxide level, breathing rate of the crew member is deviated from a predetermined breathing rate, and tissue oxygen saturation of the crew member is lower than a predetermined tissue oxygen saturation.

5. The respiratory mask of claim 1, wherein the regulator is configured to:
automatically switch to the recirculation mode when a percentage of oxygen in blood of the crew member is at least 94% and one of carbon dioxide level present in exhaled gas is lower than a predetermined carbon dioxide level and breathing rate of the crew member is higher than a predetermined breathing rate.

6. The respiratory mask of claim 1, wherein the regulator is configured to:
automatically switch to the dilution mode when at least one condition occurs, wherein the at least one condition comprises no smoke present in a cockpit, pressure level inside the cockpit is higher than a predetermined pressure level, breathing rate of the crew member is equal to a predetermined breathing rate, carbon dioxide level present in exhaled gas is equal to a predetermined carbon dioxide level, and partial pressure of oxygen present in blood of he crew member is equal to a predetermined partial pressure of oxygen.

7. The respiratory mask of claim 1, wherein the respiratory gas comprises one of oxygen during the emergency mode, a combination of exhaled gas and oxygen during the recirculation mode, and the cockpit ambient air during the dilution mode.

8. The respiratory mask of claim 1, further comprising:
a mouth and nose piece connected to the regulator;
a respiratory gas inlet and a respiratory gas outlet connected to the mouth and nose piece, wherein at least one breathing rate sensor from the plurality of sensors is disposed in the respiratory gas inlet for sensing breathing rate of the crew member and wherein at least one carbon dioxide sensor from the plurality of sensors is disposed in at least one of the respiratory gas outlet and the mouth and nose piece for sensing carbon dioxide level present in exhaled gas.

9. The respiratory mask of claim 1, further comprising:
a peripheral face seal, wherein at least one sensor from the plurality of sensors is disposed around the peripheral face seal for sensing partial pressure of oxygen present in blood of the crew member.

10. A respiratory mask for use in an aircraft, comprising:
a plurality of sensors;
a mouth and nose piece, wherein the plurality of sensors comprises at least one sensor disposed around the mouth and nose piece for sensing smoke present in a cockpit, and wherein the plurality of sensors comprises at least one sensor disposed around the mouth and nose piece for sensing a pressure level inside the cockpit;
a respiratory gas inlet and a respiratory gas outlet connected to the mouth and nose piece, wherein the plurality of sensors comprises at least one breathing rate sensor in the respiratory gas inlet for sensing breathing rate of a crew member and wherein the plurality of sensors comprises at least one carbon dioxide sensor present in the respiratory gas outlet for sensing carbon dioxide level in exhaled gas;
a peripheral face seal, wherein the plurality of sensors comprises at least one sensor disposed around the peripheral face seal for sensing partial pressure of oxygen present in blood of the crew member; and
a regulator electronically coupled to the plurality of sensors for facilitating automatic switching between operating modes to supply respiratory gas to the crew member based on output of the plurality of sensors, wherein the operating modes comprise a dilution mode, an emergency mode, and a recirculation mode.

11. A method, comprising:
receiving, from a plurality of sensors, information associated with at least one of cockpit ambient air for at least one parameter capable of affecting oxygen level and health of a crew member for at least one parameter capable of causing respiratory disorder, and providing associated output signals; and
automatically switching between operating modes of a respiratory mask to supply a respiratory gas to the crew member based on the received information, wherein the operating modes comprise a dilution mode, an emergency mode, and a recirculation mode.

12. The method of claim 11, wherein the at least one parameter capable of affecting oxygen level is selected from the group consisting of presence of smoke in a cockpit, pressure level inside the cockpit, and contaminants present in the cockpit ambient air.

13. The method of claim 11, wherein the at lest one parameter capable of causing respiratory disorder is selected from the group consisting of breathing rate of the crew member in an aircraft, carbon dioxide level present in exhaled gas of the crew ember, partial pressure of oxygen present in blood of the crew member, and tissue oxygen saturation of the crew member.

14. The method of claim 11, wherein the respiratory gas comprises one of oxygen during the emergency mode, a combination of exhaled gas and oxygen during the recirculation mode and at least one of a combination of the cockpit ambient air and the cockpit ambient air mixed with oxygen during the dilution mode.

15. The method of claim , wherein the automatically switching between the operating modes to supply the respiratory gas to the crew member based on the received information, comprises:
automatically switching to the emergency mode when at least one emergency condition occurs, wherein the at least one emergency condition comprises presence of smoke in a cockpit, pressure level inside the cockpit is higher than a predetermined pressure level, partial pressure of oxygen present in blood of the crew member is lower than a predetermined partial re of the oxygen, carbon dioxide level present in exhaled gas is higher than a predetermined carbon dioxide level, breathing rate of the screw member is deviated from a predetermined breathing rate, and tissue oxygen saturation of the crew member is lower than a predetermined tissue oxygen saturation.

16. The method of claim 11, wherein the automatically switching between the operating modes to supply the respiratory gas to the crew member based on the received information, comprises:
automatically switching to the recirculation mode when a percentage of oxygen in blood of the crew member is at least 94%, carbon dioxide level present in exhaled gas is lower than a predetermined carbon dioxide level, and breathing rate of the crew member is higher than a predetermined breathing rate.

17. The method of claim 11, wherein automatically switching between the operating modes to supply the respiratory gas to the crew member based on the received information, comprises:
automatically switching to the dilution mode when at least one condition occurs, wherein the at least one condition comprises no smoke present in a cockpit, pressure level inside the cockpit is higher than a predetermined pressure level, breathing rate of the crew member is equal to a predetermined breathing rate, carbon dioxide level present in exhaled gas is equal to a predetermined carbon dioxide level, and partial pressure of the oxygen present in blood of the crew member is equal to a predetermined partial pressure of oxygen.
